(19) United States
(12) Patent Application Publication
(10) Pub. No.: US 2010/0063112 A1
(43) Pub. Date: Mar. 11, 2010

(54) POLYMORPH III OF 4-[4-(AMINO)-3-FLUOROPHENOXY]-N-METHYLPYRIDINE-2-CARBOXAMIDE

(75) Inventors: Alfons Grunenberg, Dormagen (DE); Juergen Stiehl, Sprockhoevel (DE); Katharina Tenbieg, Dortmund (DE); Birgit Keil, Duesseldorf (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: BAYER SCHERING PHARMA AKTIENGESELLSCHAFT

(21) Appl. No.: 12/514,129

(22) PCT Filed: Nov. 3, 2007
(86) PCT No.: PCT/EP2007/009545

§ 371 Date: Nov. 9, 2009

(30) Foreign Application Priority Data
Nov. 9, 2006 (EP) 06023341.8

(51) Int. Cl. A61K 31/44; C07D 213/78; A61P 35/00

(52) U.S. Cl. 514/350; 546/298

(57) ABSTRACT

The present invention relates to the polymorph III of 4-[4-({[4-chloro-3-(trifluoromethyl)phenyl]carbamoyl}amino)-3-fluorophenoxy]-N-methylpyridine-2-carboxamide, to processes for its preparation, to pharmaceutical compositions comprising it and to its use in the control of disorders.

The present invention relates to the polymorph III of 4-[4-({[4-chloro-3-(trifluoromethyl)phenyl]carbamoyl}amino)-3-fluorophenoxy]-N-methylpyridine-2-carboxamide, to processes for its preparation, to pharmaceutical compositions comprising it and to its use in the control of disorders.

4-[4-({[4-chloro-3-(trifluoromethyl)phenyl]carbamoyl}amino)-3-fluorophenoxy]-N-methylpyridine-2-carboxamide is mentioned in WO 2005/009961 and corresponds to the compound of the formula (I):

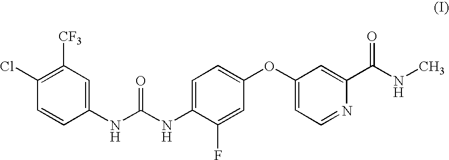

WO 2005/009961 describes the compound of formula (I) as an inhibitor of the enzyme Raf kinase which may be used for the treatment of disorders in which angiogenesis and/hyper-proliferation plays an important role, for example in tumor growth and cancer.

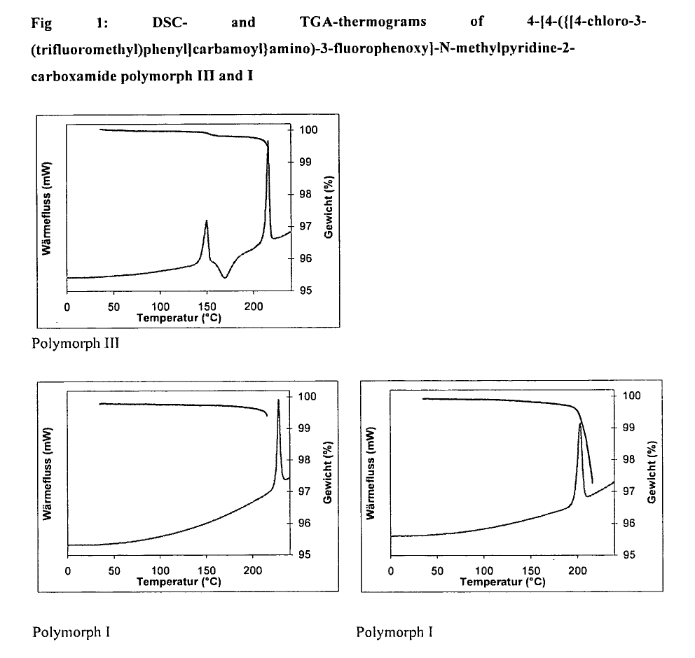

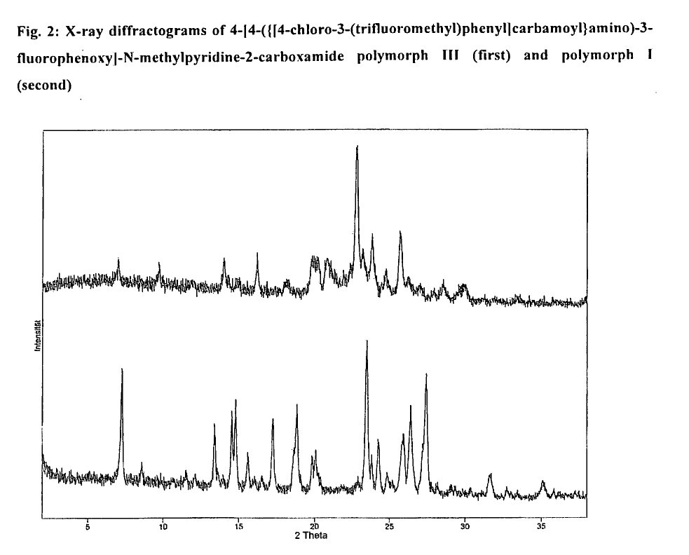

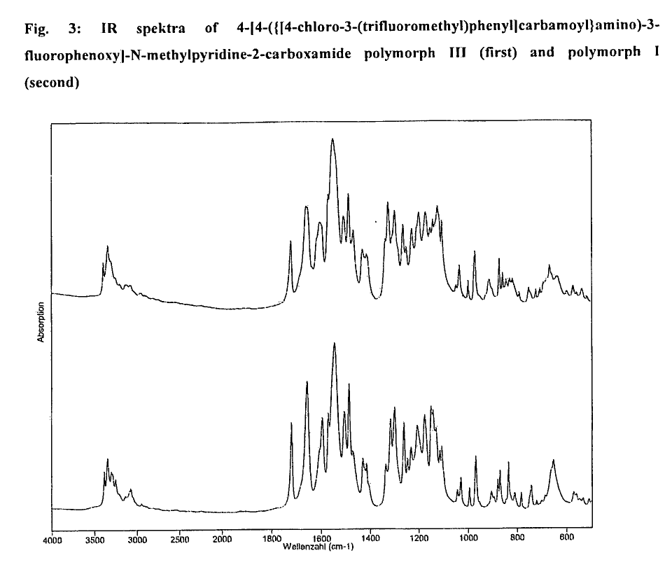

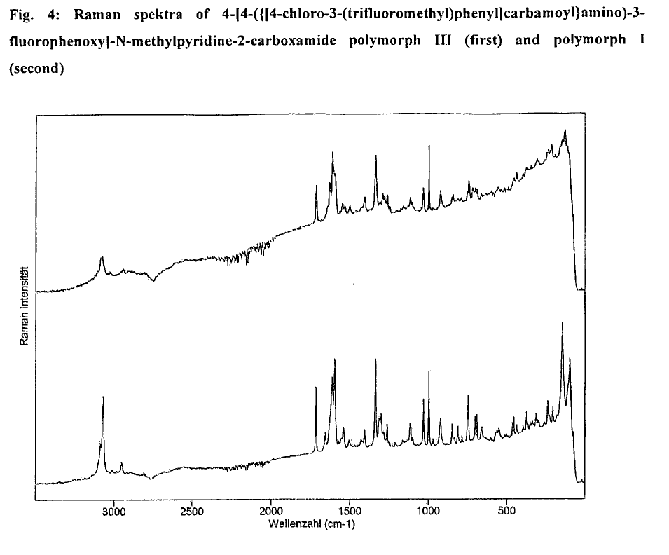

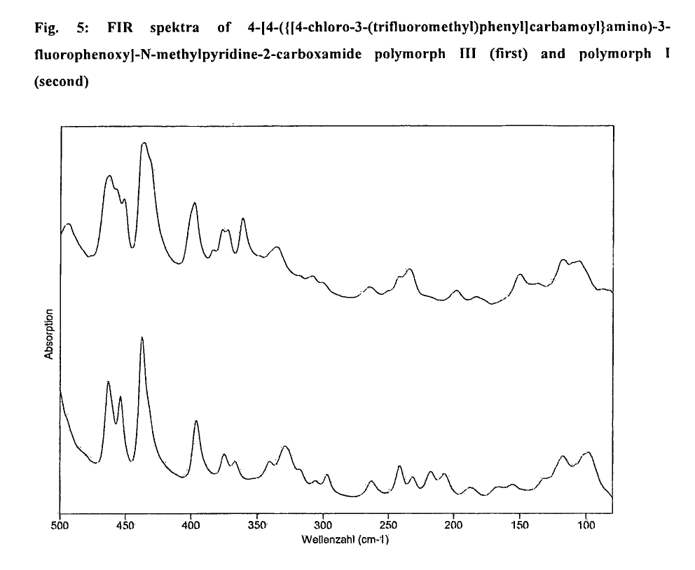

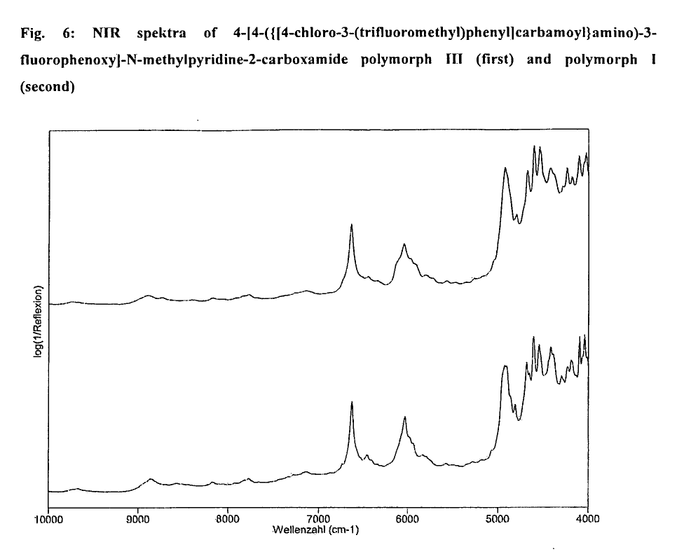

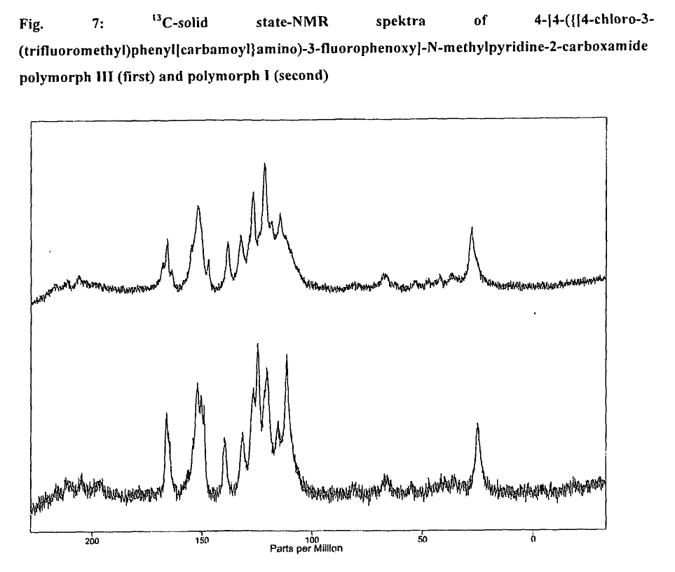

The compound of the formula (I) is prepared in the manner described in WO 2005/009961 and corresponds to a polymorph which in the following is named as polymorph I having a melting point of 186-206° C., a characteristic X-ray diffractogram, IR spectrum, Raman spectrum, FIR spectrum, NIR spectrum and a ¹³C-solid state-NMR spectrum (Tab. 2-7, FIGS. 2-7).

The present invention provides a new polymorph of the compound of the formula (I), which melts at 141° C. and is called polymorph III.

In comparison to the polymorph I of the compound of the formula (I), polymorph III has a clearly differentiable X-ray diffractogram, IR spectrum, Raman spectrum, FIR spectrum, NIR spectrum and ¹³C-solid state NMR spectrum (FIGS. 2-7).

Surprisingly the new polymorph III of the compound of formula (I) has a high solubility in water and in organic solvents.

The inventive compound of the formula (I) in the polymorph III is used in high purity in pharmaceutical formulations. For reasons of stability, a pharmaceutical formulation comprises the compound of the formula (I) in the polymorph III mainly and no significant fractions of another form of the compound of the formula (I), for example of another polymorph or pseudopolymorph of the compound of the formula (I). The pharmaceutical composition preferably contains more than 90 percent by weight, more preferably more than 95 percent by weight, of the compound of the formula (I) in the polymorph III related to the total amount of the compound of the formula (I) present in the composition.

### Method for Treatment:

The present invention also relates to a method for using the compound of the formula (I) in the polymorph III and compositions thereof, to treat mammalian hyper-proliferative disorders. This method comprises administering to a mammal in need thereof, including a human, an amount of a compound of the formula (I) in the polymorph III of this invention or composition thereof, which is effective to treat the disorder. Hyper-proliferative disorders include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypothalamic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal/nasopharyngeal/oropharyngeal cancer, and lip and oral cavity cancer.

Lymphomas include, but are not limited to AIDS-related lymphoma, non-Hodgkin's lymphoma, cutaneous T-cell lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

Based upon standard laboratory techniques known to evaluate compounds useful for the treatment of hyper-proliferative disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The present invention further provides the use of the compound of the formula (I) in the polymorph III for the preparation of a pharmaceutical compositions for the treatment of the aforesaid disorders.

Combination with Other Pharmaceutical Agents:

The compound of the formula (I) in the polymorph III of this invention can be administered as the sole pharmaceutical agent or in combination with one or more other pharmaceutical agents where the combination causes no unacceptable adverse effects. For example, the compound of the formula (I) in the polymorph III of this invention can be combined with known anti-hyper-proliferative or other indication agents, and the like, as well as with admixtures and combinations thereof.

Optional anti-hyper-proliferative agents which can be added to the compositions include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the compositions of the invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the compositions of the invention include but are not limited to other anti-cancer agents such as epothilone and its derivatives, irinotecan, raloxifen and topotecan.

Generally, the use of cytotoxic and/or cytostatic agents in combination with a compound or composition of the present invention will serve to:
(1) yield better efficacy in reducing the growth of a tumor or even eliminate the tumor as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

“Combination” mean for the purposes of the invention not only a dosage form which contains all the components (so-called fixed combinations), and combination packs containing the components separate from one another, but also components which are administered simultaneously or sequentially, as long as they are employed for the prophylaxis or treatment of the same disease.

The active ingredients of the combination according to the invention can be converted in a known manner into the usual formulations, which may be liquid or solid formulations. Examples are tablets, coated tablets, pills, capsules, granules, aerosols, syrups, emulsions, suspensions, solutions.

Since the combination according to the invention is well tolerated and in some cases is effective even in low dosages, a wide range of formulation variants is possible. Thus, one possibility is to formulate the individual active ingredients of the combination according to the invention separately. In this case, it is not absolutely necessary for the individual active ingredients to be taken at the same time; on the contrary, sequential intake may be advantageous to achieve optimal effects. It is appropriate with such separate administration to combine the formulations of the individual active ingredients, for example tablets or capsules, simultaneously together in a suitable primary packaging. The active ingredients are present in the primary packaging in each case in separate containers which may be, for example, tubes, bottles or blister packs. Such separate packaging of the components in the joint primary packaging is also referred to as a kit.

Further formulation variants which are suitable and preferred for the combination according to the invention are also fixed combinations. “Fixed combination” is intended here to mean pharmaceutical forms in which the components are present together in a fixed ratio of amounts. Such fixed combinations may be, for example, in the form of oral solutions, but they are preferably solid oral pharmaceutical preparations, e.g. capsules or tablets.

### Pharmaceutical Compositions:

This invention also relates to pharmaceutical compositions containing the compound of the formula (I) in the polymorph III of the present invention. These compositions can be utilized to achieve the desired pharmacological effect by administration to a patient in need thereof. A patient, for the purpose of this invention, is a mammal, including a human, in need of treatment for the particular condition or disease. Therefore, the present invention includes pharmaceutical compositions which are comprised of a pharmaceutically acceptable carrier and a pharmaceutically effective amount of a compound of the formula (I) in the polymorph of the present invention. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is that amount which produces a result or exerts an influence on the particular condition being treated. The compound of the formula (I) in the polymorph III of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

For oral administration, the compound of the formula (I) in the polymorph III can be formulated into solid or liquid preparations such as solid dispersion, capsules, pills, tablets, troches, lozenges, melts, powders, solutions, suspensions, or emulsions, and may be prepared according to methods known to the art for the manufacture of pharmaceutical compositions. The solid unit dosage forms can be a capsule which can be of the ordinary hard- or soft-shelled gelatin type containing, for example, surfactants, lubricants, and inert fillers such as lactose, sucrose, calcium phosphate, and corn starch.

In another embodiment, the compound of the formula (I) in the polymorph III of this invention may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

The compound of the formula (I) in the polymorph III of this invention may also be administered parenterally, that is, subcutaneously, intravenously, intraocularly, intrasynovially, intramuscularly, or interperitoneally, as injectable dosages of the compound in a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjutants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A compositions of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such material is, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations which are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The pharmaceutical compositions of this invention may also be in the form of a solid dispersion. The solid dispersion may be a solid solution, glass solution, glass suspension, amorphous precipitation in a crystalline carrier, eutectic or monotecic, compound or complex formation and combinations thereof.

An aspect of the invention of particular interest is a pharmaceutical composition comprising a solid dispersion, wherein the matrix comprises a pharmaceutically acceptable polymer, such as polyvinylpyrrolidone, vinylpyrrolidone/vinylacetate copolymer, polyalkylene glycol (i.e. polyethylene glycol), hydroxyalkyl cellulose (i.e. hydroxypropyl cellulose), hydroxyalkyl methyl cellulose (i.e. hydroxypropyl methyl cellulose), carboxymethyl cellulose, sodium carboxymethyl cellulose, ethyl cellulose, polymethacrylates, polyvinyl alcohol, polyvinyl acetate, vinyl alcohol/vinyl acetate copolymer, polyglycolized glycerides, xanthan gum, carrageenan, chitosan, chitin, polydextran, dextrin, starch and proteins.

Another aspect of the invention is a pharmaceutical composition comprising a solid dispersion, wherein the matrix comprises a sugar and/or sugar alcohol and/or cyclodextrin, for example sucrose, lactose, fructose, maltose, raffinose, sorbitol, lactitol, mannitol, maltitol, erythritol, inositol, trehalose, isomalt, inulin, maltodextrin, β-cyclodextrin, hydroxypropyl-β-cyclodextrin or sulfobutyl ether cyclodextrin.

Additional suitable carriers that are useful in the formation of the matrix of the solid dispersion include, but are not limited to alcohols, organic acids, organic bases, amino acids, phospholipids, waxes, salts, fatty acid esters, polyoxyethylene sorbitan fatty acid esters, and urea.

The solid dispersion of the compound of formula (I) in the polymorph III in the matrix may contain certain additional pharmaceutical acceptable ingredients, such as surfactants, fillers, disintegrants, recrystallization inhibitors, plasticizers, defoamers, antioxidants, detackifier, pH-modifiers, glidants and lubricants.

The solid dispersion of the invention is prepared according to methods known to the art for the manufacture of solid dispersions, such as fusion/melt technology, hot melt extrusion, solvent evaporation (i.e. freeze drying, spray drying or layering of powders of granules), coprecipitation, supercritical fluid technology and electrostatic spinning method.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients which can be used as appropriate to formulate the composition for its intended route of administration include:
acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal);
aerosol propellants (examples include but are not limited to carbon dioxide, CCl₂F₂, F₂ClC—CClF₂ and CClF₃) air displacement agents (examples include but are not limited to nitrogen and argon);
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfate, sodium formaldehyde sulfoxylate, sodium metabisulfite);
binding materials (examples include but are not limited to block polymers, natural and synthetic rubber, polyacrylates, polyurethanes, silicones, polysiloxanes and styrene-butadiene copolymers);
buffering agents (examples include but are not limited to potassium metaphosphate, dipotassium phosphate, sodium acetate, sodium citrate anhydrous and sodium citrate dihydrate)
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)
chelating agents (examples include but are not limited to edetate disodium and edetic acid)
colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);
clarifying agents (examples include but are not limited to bentonite);
emulsifying agents (examples include but are not limited to acacia, cetomacrogol, cetyl alcohol, glyceryl monostearate, lecithin, sorbitan monooleate, polyoxyethylene 50 monostearate);
encapsulating agents (examples include but are not limited to gelatin and cellulose acetate phthalate)
flavorants (examples include but are not limited to anise oil, cinnamon oil, cocoa, menthol, orange oil, peppermint oil and vanillin);
humectants (examples include but are not limited to glycerol, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)
plasticizers (examples include but are not limited to diethyl phthalate and glycerol);
solvents (examples include but are not limited to ethanol, corn oil, cottonseed oil, glycerol, isopropanol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan mono-palmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);
sweetening agents (examples include but are not limited to aspartame, dextrose, glycerol, mannitol, propylene glycol, saccharin sodium, sorbitol and sucrose);
tablet anti-adherents (examples include but are not limited to magnesium stearate and talc);
tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide);
tablet polishing agents (examples include but are not limited to carnauba wax and white wax);
thickening agents (examples include but are not limited to beeswax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, polyvinyl pyrrolidone, sodium alginate and tragacanth); and
wetting agents (examples include but are not limited to heptadecaethylene oxycetanol, lecithin, sorbitol monooleate, polyoxyethylene sorbitol monooleate, and polyoxyethylene stearate).

It is believed that one skilled in the art, utilizing the preceding information, can utilize the present invention to its fullest extent. Nevertheless, the following are examples of pharmaceutical formulations that can be used in the method of the present invention. They are for illustrative purposes only, and are not to be construed as limiting the invention in any way.

Pharmaceutical compositions according to the present invention can be illustrated as follows:

Sterile IV Solution: A 5 mg/ml solution of the desired compound of this invention is made using sterile, injectable water, and the pH is adjusted if necessary. The solution is diluted for administration to 1-2 mg/ml with sterile 5% dextrose and is administered as an IV infusion over 60 minutes.
Lyophilized powder for IV administration: A sterile preparation can be prepared with (i) 100-1000 mg of the desired compound of this invention as a lyophilized powder, (ii) 32-327 mg/ml sodium citrate, and (iii) 300-3000 mg Dextran 40. The formulation is reconstituted with sterile, injectable saline or dextrose 5% to a concentration of 10 to 20 mg/ml, which is further diluted with saline or dextrose 5% to 0.2-0.4 mg/ml, and is administered either IV bolus or by IV infusion over 15-60 minutes.
Intramuscular suspension: The following solution or suspension can be prepared, for intramuscular injection:
50 mg/ml of the desired, water-insoluble compound of this invention
5 mg/ml sodium carboxymethylcellulose
4 mg/ml TWEEN 80
9 mg/ml sodium chloride
9 mg/ml benzyl alcohol
   Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.
   Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.
   Tablets: A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.
   Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### Dosage of the Pharmaceutical Compositions of the Present Invention:

Based upon standard laboratory techniques known to evaluate compounds useful for the treatment of hyper-proliferative disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg, and preferably from about 0.01 mg/kg to about 20 mg/kg body weight per day. A unit dosage may contain from about 0.5 mg to about 1500 mg of active ingredient, and can be administered one or more times per day. The daily dosage for administration by injection, including intravenous, intramuscular, subcutaneous and parenteral injections, and use of infusion techniques will preferably be from 0.01 to 200 mg/kg of total body weight. The daily rectal dosage regimen will preferably be from 0.01 to 200 mg/kg of total body weight. The daily vaginal dosage regimen will preferably be from 0.01 to 200 mg/kg of total body weight. The daily topical dosage regimen will preferably be from 0.1 to 200 mg administered between one to four times daily. The transdermal concentration will preferably be that required to maintain a daily dose of from 0.01 to 200 mg/kg. The daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### Process for Preparing

The invention further provides a process for preparing the compound of the formula (I) in the polymorph III by treating the monohydrate of the compound of formula (I) at higher temperatures until quantitative conversion to polymorph III is achieved. The monohydrate of the compound of formula (I) is described in the European patent application EP 06021296.6 and corresponds to example 1 of the present invention.

Preference is given to a process wherein the monohydrate of the compound of formula (I) is tempered for e.g. 1 to 2 h at 80 to 120° C. More preferably the monohydrate of the compound of formula (I) is tempered for 1 h at 100° C. followed by 15 min at 110° C.

The processes are generally carried out at atmospheric pressure. However, it is also possible to work at elevated pressure or at reduced pressure (for example in a range of from 0.5 to 5 bar).

It is believed that one skilled in the art, using the preceding information and information available in the art, can utilize the present invention to its fullest extent.

It should be apparent to one of ordinary skill in the art that changes and modifications can be made to this invention without departing from the spirit or scope of the invention as it is set forth herein.

All publications, applications and patents cited above and below are incorporated herein by reference.

The weight data in the tests and examples which follow are, unless stated otherwise, percentages by weight; parts are parts by weight. Solvent ratios, dilution ratios and concentration data of liquid/liquid solutions are based on each case on the volume.

### WORKING EXAMPLES

The thermograms are obtained using a DSC 7 or Pyris-1 differential scanning calorimeter and TGA 7 thermogravimetric analyzer from Perkin-Elmer. The X-ray diffractograms are registered in a Stoe transmission diffractometer. The IR, FIR, NIR and Raman spectra are recorded using IFS 66v (IR, FIR). IFS 28/N (NIR) and RFS 100 (Raman) Fourier spectrometers from Bruker. The ¹³C-solid state NMR spectra are recorded using the NMR spectrometer DRX400 from Bruker.

### Example 1

### Preparation of 4-[4-({[4-chloro-3-(trifluoromethyl)phenyl]carbamoyl}amino)-3-fluorophenoxy]-N-methylpyridine-2-carboxamide monohydrate

### Example 1.1

400 mg of 4-[4-({[4-chloro-3-(trifluoromethyl)phenyl]carbamoyl}amino)-3-fluorophenoxy]-N-methylpyridine-2-carboxamide in the polymorph I, prepared as described in WO 2005/009961, are dissolved in acetone and the solution is filtered. Water is added to one fourth of the filtrate until precipitation. The precipitate is filtered and dried at room temperature under ambient humidity. The sample is tested gravimetrically and corresponds to the monohydrate of the title compound.

### Example 1.2

400 mg of 4-[4-({[4-chloro-3-(trifluoromethyl)phenyl]carbamoyl}amino)-3-fluorophenoxy]-N-methylpyridine-2-carboxamide in the polymorph T, prepared as described in WO 2005/009961, are dissolved in 50 ml of ethanol and the solution is filtered. One fourth of the solution is stayed in the freezer for crystallization at about −20° C. until the solvent is evaporated. The residue is tested by X-ray diffractometry and corresponds to the monohydrate of the title compound.

### Example 1.3

100 mg of 4-[4-({[4-chloro-3-(trifluoromethyl)phenyl]carbamoyl}amino)-3-fluorophenoxy]-N-methylpyridine-2-carboxamide in the polymorph I, prepared as described in WO 2005/009961, are suspended in 2 ml of a mixture of acetonitril-water (1:1) and shaken at 25° C. After one week the suspension is filtered and the residue is dried at room temperature and ambient humidity. The residue is tested gravimetrically and corresponds to the monohydrate of the title compound.

### Example 1.4

100 mg of 4-[4-({[4-chloro-3-(trifluoromethyl)phenyl]carbamoyl}amino)-3-fluorophenoxy]-N-methylpyridine-2-carboxamide in the polymorph I, prepared as described in WO 2005/009961, are suspended in 2 ml of a mixture of tetrahydrofuran-water (1:1) and stirred at 10° C. After two weeks the suspension is filtered and the residue is dried at room temperature and ambient humidity. The residue is tested by X-ray diffractometry and corresponds to the monohydrate of the title compound.

### Example 2

### Preparation of 4-[4-({[4-chloro-3-(trifluoromethyl)phenyl]carbamoyl}amino)-3-fluorophenoxy]-N-methylpyridine-2-carboxamide in the polymorph III

### Example 2.1

3.5 g of the compound according to example 1 are tempered for 60 min at 100° C. and further 15 min at 110° C. After cooling to room temperature the product is tested by X-ray diffractometry and corresponds to the title compound.

### Example 2.2

9 mg of the compound according to example 1 are heated to 100° C. in a Thermogravimetric Analyzer (TGA) and are tempered for 10 min at that temperature. After cooling to room temperature the product is tested by X-ray diffractometry and corresponds to the title compound.

### Example 2.3

50 mg of the compound according to example 1 are tempered for 10 min at 110° C. After cooling to room temperature the product is tested by X-ray diffractometry and corresponds to the title compound.

TABLE 1
Differential Scanning Calorimetry and Thermogravimetry

| | Polymorph I | Polymorph III |
|---|---|---|
| Melting point [° C.] | 186-206 | 141 |
| Loss in mass [% by wt.] | <0.4 | <0.4 |

TABLE 2
X-ray diffractometry
Peak maxima [2 Theta]

| Polymorph I | Polymorph III |
|---|---|
| 7.2 | 7.0 |
| 7.3 | 9.7 |
| 8.6 | 11.8 |
| 10.7 | 14.0 |
| 11.5 | 15.0 |
| 12.1 | 16.2 |
| 13.4 | 18.1 |
| 13.6 | 19.8 |
| 14.0 | 20.1 |
| 14.5 | 20.2 |
| 14.8 | 20.8 |
| 15.6 | 21.0 |
| 16.0 | 21.3 |
| 16.5 | 21.9 |
| 17.2 | 22.3 |
| 18.6 | 22.8 |
| 18.8 | 23.1 |
| 19.1 | 23.4 |
| 19.8 | 23.8 |
| 20.1 | 24.4 |
| 20.2 | 25.7 |
| 20.4 | 26.2 |
| 21.8 | 27.0 |
| 22.9 | 27.9 |
| 23.5 | 28.6 |
| 23.8 | 29.6 |
| 24.2 | 29.9 |
| 24.9 | 31.2 |
| 25.2 | 33.5 |
| 25.9 | |
| 26.0 | |
| 26.4 | |
| 26.6 | |
| 27.2 | |
| 27.4 | |
| 28.2 | |
| 29.1 | |
| 29.4 | |
| 30.4 | |
| 30.9 | |
| 31.6 | |
| 32.7 | |
| 33.0 | |
| 33.4 | |
| 35.1 | |
| 35.3 | |
| 35.8 | |
| 36.1 | |
| 36.6 | |
| 37.3 | |

TABLE 3
IR spectroscopy
Peak maxima [cm⁻¹]

| Polymorph I | Polymorph III |
|---|---|
| 512 | 514 |
| 535 | 534 |
| 563 | 569 |
| 572 | 595 |
| 654 | 631 |
| 722 | 664 |
| 744 | 704 |
| 785 | 720 |
| 811 | 749 |
| 836 | 788 |
| 871 | 815 |
| 880 | 827 |
| 906 | 842 |
| 970 | 856 |
| 996 | 869 |
| 1030 | 909 |
| 1044 | 968 |
| 1108 | 996 |
| 1116 | 1301 |
| 1131 | 1045 |
| 1143 | 1102 |
| 1151 | 1119 |
| 1176 | 1138 |
| 1207 | 1150 |
| 1233 | 1168 |
| 1246 | 1196 |
| 1261 | 1225 |
| 1300 | 1247 |
| 1317 | 1260 |
| 1336 | 1295 |
| 1416 | 1322 |
| 1431 | 1333 |
| 1471 | 1411 |
| 1487 | 1428 |
| 1506 | 1465 |
| 1546 | 1484 |
| 1572 | 1504 |
| 1596 | 1548 |
| 1657 | 1568 |
| 1720 | 1602 |
| 3077 | 1656 |
| 3255 | 1718 |
| 3306 | 3078 |
| 3350 | 3136 |
| 3389 | 3342 |
| | 3396 |

TABLE 4
Raman spectroscopy
Peak maxima [cm⁻¹]

| Polymorph I | Polymorph III |
|---|---|
| 85 | 112 |
| 105 | 137 |
| 151 | 220 |
| 213 | 439 |
| 245 | 456 |
| 317 | 691 |
| 340 | 718 |
| 352 | 742 |
| 375 | 844 |
| 397 | 924 |
| 438 | 997 |
| 457 | 1033 |
| 465 | 1115 |
| 551 | 1262 |
| 659 | 1291 |
| 691 | 1336 |
| 701 | 1406 |
| 746 | 1502 |
| 786 | 1550 |
| 811 | 1598 |
| 849 | 1613 |
| 921 | 1632 |
| 970 | 1717 |
| 997 | 2023 |
| 1030 | 2043 |
| 1099 | 2060 |
| 1111 | 2074 |
| 1116 | 2092 |
| 1209 | 2136 |
| 1261 | 2146 |
| 1284 | 2157 |
| 1300 | 2171 |
| 1314 | 2185 |
| 1336 | 2221 |
| 1405 | 2246 |
| 1427 | 2283 |
| 1504 | 3079 |
| 1541 | |
| 1597 | |
| 1613 | |
| 1657 | |
| 1717 | |
| 2951 | |
| 3071 | |
| 3090 | |

TABLE 5
FIR spectroscopy
Peak maxima [cm⁻¹]

| Polymorph I | Polymorph III |
|---|---|
| 99 | 106 |
| 117 | 117 |
| 155 | 150 |
| 166 | 183 |
| 187 | 198 |
| 207 | 234 |
| 217 | 242 |
| 231 | 265 |
| 241 | 308 |
| 263 | 336 |
| 297 | 361 |
| 306 | 373 |
| 318 | 377 |
| 329 | 384 |
| 341 | 398 |
| 367 | 436 |
| 375 | 452 |
| 396 | 463 |
| 438 | 494 |
| 454 | |
| 463 | |

TABLE 6
NIR spectroscopy
Peak maxima [cm⁻¹]

| Polymorph I | Polymorph III |
|---|---|
| 4041 | 4028 |
| 4098 | 4106 |
| 4190 | 4183 |
| 4230 | 4238 |
| 4296 | 4421 |
| 4414 | 4538 |
| 4542 | 4600 |
| 4604 | 4675 |
| 4681 | 4796 |
| 4808 | 4924 |
| 4924 | 6045 |
| 6033 | 6642 |
| 6632 | |
| 8858 | |

TABLE 7
¹³C-solid state-NMR spectroscopy
Peak maxima [ppm]

| Polymorph I | Polymorph III |
|---|---|
| 25 | 28 |
| 105 | 115 |
| 112 | 119 |
| 116 | 122 |
| 121 | 127 |
| 125 | 133 |
| 127 | 139 |
| 131 | 147 |
| 139 | 152 |
| 149 | 155 |
| 150 | 157 |
| 152 | 164 |
| 166 | 168 |

1. A compound of the formula (I) 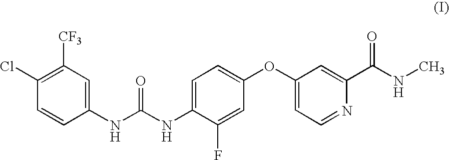
in the polymorph III.

2. The compound of claim 1 which shows in the X-ray diffractometry a peak maximum of the 2 Theta angel of 16.2.

3. The compound of claim 1 which shows in the IR spectrum a peak maximum of 856 cm⁻¹.

4. A process for the preparation of the compound of the formula (I) in the polymorph III wherein the monohydrate of the compound of formula (I) is tempered at a higher temperature until quantitative conversion into polymorph III.

5. A compound of the formula (I) in the polymorph III of claim 1 for the treatment of hyper-proliferative disorders.

6. A compound of the formula (I) in the polymorph III of claim 1 for the treatment of solid tumors, lymphomas, sarcomas, leukemias, cancers of the breast, respiratory tract, brain, reproductive organs, digestive tract, urinary tract, eye, liver, skin, head and neck, thyroid and/or parathyroid.

7. A use of the compound of the formula (I) in the polymorph III of claim 1 for the preparation of a pharmaceutical composition for the treatment of hyper-proliferative disorders.

8. The use of claim 7 for the treatment of solid tumors, lymphomas, sarcomas, leukemias, cancers of the breast, respiratory tract, brain, reproductive organs, digestive tract, urinary tract, eye, liver, skin, head and neck, thyroid and/or parathyroid.

9. A pharmaceutical composition comprising the compound of the formula (I) in the polymorph III of claim 1 mainly, no significant fractions of another form of 4-[4-({[4-chloro-3-(trifluoromethyl)phenyl]carbamoyl}amino)-3-fluorophenoxy]-N-methylpyridine-2-carboxamide and one or more inert, nontoxic, pharmaceutically suitable excipients.

10. The pharmaceutical composition of claim 9 containing more than 90 percent by weight of the compound of the formula (I) in the polymorph III related to the total amount of the compound of the formula (I) in the polymorph III present in the composition.

11. The pharmaceutical composition of claim 9 for the treatment of disorders.

12. A method for treating hyper-proliferative disorders using an effective amount of the compound of the formula (I) in the polymorph III of claim 1.

13. A combination comprising the compound of the formula (I) in the polymorph III of claim 1 and one or more other pharmaceutical agents.

14. The combination of claim 13 wherein the one or more other pharmaceutical agents are cytotoxic agents, signal transduction inhibitors, anti-cancer agents, or antiemetics.

15. The pharmaceutical composition claim 9 comprising one or more other pharmaceutical agents.

16. The pharmaceutical composition of claim 15 wherein the one or more other pharmaceutical agents are anti-hyper-proliferative agents, cytotoxic agents, signal transduction inhibitors, anti-cancer agents and/or antiemetics.
